SEQUENCE LISTING (4 sequences)

SEQ ID NO: 1 - DNA, 894 bp, human
gggagtggagtgaggggtaacaagatggcgaccgagacggtggagctccataagctaaagcttgccgaactaaagcaagaatgtcttgctcgtggtttggagaccaagggaataaagcaagatcttatccacagactccaggcatatcttgaagaacatgctgaagaggaggcaaatgaagaagatgtactgggagatgaaacagaggaagaagaaacaaagcccattgagctccctgtcaaagaggaagaaccccctgaaaaaactgttgatgtggcagcagagaagaaagtggtgaaaattacatctgaaataccacagactgagagaatgcagaagagggctgaacgattcaatgtacctgtgagcttggagagtaagaaagctgctcgggcagctaggtttgggatttcttcagttccaacaaaaggtctgtcatctgataacaaacctatggttaacttggataagctgaaggaaagagctcaaagatttggtttgaatgtctcttcaatctccagaaagtctgaagatgatgagaaactgaaaaagaggaaggagcgatttgggattgtcacaagttcagctggaactggaaccacagaggatacagaggcaaagaagaggaaaagagcagagcgctttgggattgcctgatgaaaagttcctgatactttctgttctccagtgttttccatttctctccttcttcttggtcacatatatgcctaaatgcacagtcatgtgcctacgtcctgcctcgcaatgagggagcatgtaccccaggtacatccatgaactgcggcagcagtttgacttattgctgtttcagctttaaggttgttgtgtttttgtttttgattatgttgcttgttaataaaaaaaaatagaaaa

SEQ ID NO: 2 - protein, 210 aa, human
MATETVELHKLKLAELKQECLARGLETKGIKQDLIHRLQAYLEEHAEEEANEEDVLGDETEEEETKPIELPVKEEEPPEKTVDVAAEKKVVKITSEIPQTERMQKRAERFNVPVSLESKKAARAARFGISSVPTKGLSSDNKPMVNLDKLKERAQRFGLNVSSISRKSEDDEKLKKRKERFGIVTSSAGTGTTEDTEAKKRKRAERFGIA

SEQ ID NO: 3 - DNA, 873 bp, Artificial Sequence
  PCR Product
tggagtgaggggtaacaagatggcgaccgagacggtggagctccataagctaaagcttgccgaactaaagcaagaatgtcttgctcgtggtttggagaccaagggaataaagcaagatcttatccacagactccaggcatatcttgaagaacatgctgaagaggaggcaaatgaagaagatgtactgggagatgaaacagaggaagaagaaacaaagcccattgagctccctgtcaaagaggaagaaccccctgaaaaaactgttgatgtggcagcagagaagaaagtggtgaaaattacatctgaaataccacagactgagagaatgcagaagagggctgaacgattcaatgtacctgtgagcttggagagtaagaaagctgctcgggcagctaggtttgggatttcttcagttccaacaaaaggtctgtcatctgataacaaacctatggttaacttggataagctgaaggaaagagctcaaagatttggtttgaatgtctcttcaatctccagaaagtctgaagatgatgagaaactgaaaaagaggaaggagcgatttgggattgtcacaagttcagctggaactggaaccacagaggatacagaggcaaagaagaggaaaagagcagagcgctttgggattgcctgatgaaaagttcctgatactttctgttctccagtgttttccatttctctccttcttcttggtcacatatatgcctaaatgcacagtcatgtgcctacgtcctgcctcgcaatgagggagcatgtaccccaggtacatccatgaactgcggcagcagtttgacttattgctgtttcagctttaaggttgttgtgtttttgtttttgattatgttgcttgttaat

SEQ ID NO: 4 - DNA, 690 bp, human
caggggcagcagtgattatctgaactcggatctttaaaattgtggtagctctaaagctgatgatgtctggttaggaagtggctcttgcccgccccagccccaccgccagttccttaagcccgccccatgcccctcccagcttcctcctcatgttcatcggttttttcagggctcccttcaacgctcccctctcagtatttaggtcaccactccctcggcgcccctttcgcctcccaccatttttcctcagcaacccttacagtctttgcagctcctacctgccagctcagatccccgtccggctatgggcgcggcgccggctaccacacctgaagtctccaggaagtaacgcctctccttctgcccctttcctgttggaggaacagaatcagcgctgccaccacccattggttggtggtctgtaatgcagaagcacagttggttgccatttctgtcgttcgcaagatacagtgcccgcccctctcccagttccaccttttgaaagaggtggggcaagctgcctagagaagtgagagcgacgtcagctattgaccaatgggaagagctgatggtatggcgtgggagcaagagtgacaacgattggtcagccttgcatctctacgcctaaggcgggaactcctggaggcggaggccgcgggtggggggagtggagtgaggggtaacaagatg

(19) United States
(12) Patent Application Publication
(10) Pub. No.: US 2006/0234290 A1
(43) Pub. Date: Oct. 19, 2006

(54) Novel genes and expression products therefrom

(76) Inventors: Ching Ming Chung, Singapore (SG); Lily Chan, Singapore (SG); Keli Ou, Singapore (SG); Shao-En Ong, Singapore (SG); Teck Keong Seow, Singapore (SG); Cynthia Rosa M.Y. Liang, Singapore (SG); Meng Ling Choong, Singapore (SG); Li Kiang Tan, Singapore (SG)

Correspondence Address: BIRCH STEWART KOLASCH & BIRCH, PO BOX 747, FALLS CHURCH, VA, 22040-0747, US

(21) Appl. No.: 11/476,634

(22) Filed: Jun. 29, 2006

Related U.S. Application Data
(62) Division of application No. 09/788,476, filed on Feb. 21, 2001.
(60) Provisional application No. 60/185,116, filed on Feb. 25, 2000.

(51) Int. Cl. C12Q 1/68; C07H 21/04; A61K 48/00; C07K 14/82

(52) U.S. Cl. 435/6; 536/23.5; 530/350; 514/44

(57) ABSTRACT

The present invention relates generally to the identification of the products of gene expression in cancerous tissue or other tissue associated with an aberrant medical condition. Identification of such expression products enables the development of a range of diagnostic and therapeutic agents. In one embodiment of the present invention, a gene is differentially or preferentially expressed in cancerous tissue relative to normal tissue. Identification of the expression product of the gene and of the gene itself provides a means of developing diagnostic and therapeutic agents for the treatment, prophylaxis, and diagnosis of the cancerous condition in which the gene is differentially or preferentially expressed.

This application is a division of application Ser. No. 09/788,476, which was filed on Feb. 21, 2001. This application claims priority under 35 USC § 119 to provisional application Ser. No. 60/185,116, which was filed on Feb. 25, 2000. The entire contents of each of application Ser. Nos. 09/788,476 and 60/185,116 are expressly incorporated by reference in the present application.

### FIELD OF THE INVENTION

The present invention relates generally to the identification of the products of gene expression in cancerous tissue or other tissue associated with an aberrant medical condition. The identification of such expression products enables the development of a range of diagnostic and therapeutic agents.

In one embodiment, a gene is differentially or preferentially expressed in cancerous tissue relative to normal tissue. The identification of the expression product of the gene and of the gene itself provides a means of developing diagnostic and therapeutic agents for the treatment, prophylaxis and diagnosis of the cancerous condition in which the gene is differentially or preferentially expressed. In another embodiment, the gene is involved in transcriptional control and hence modulating gene expression is contemplated as a means of modulating cell regulation.

### BACKGROUND OF THE INVENTION

The increasing sophistication of recombinant DNA techniques is greatly facilitating research and development in the medical and allied health fields. This is particularly the case as the human genome sequencing project nears completion. However, in addition to elucidating the nucleotide sequence of the human genome, there is a requirement to undertake functional analyses of particular nucleotide sequences, especially those forming transcription units, i.e. genes.

A functional analysis involves the determination of expression patterns. For example, some genes may be expressed preferentially or exclusively during particular disease conditions such as cancer or autoimmune conditions. The identification of such genes provides a basis for developing a range of diagnostic and therapeutic agents aimed, for example, at identifying expression of the gene and/or developing protocols for down-regulating expression of the gene.

In work leading up to the present invention, the inventors sought to identify genes differentially or preferentially expressed in human hepatocellular carcinoma. This is one of the most frequently encountered malignancies affecting Asia and China (Schafer and Sorrell, 1999).

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A novel protein, HCC-1, is identified from the HCC-M cell line through a 2D gel electrophoresis and mass spectrometry analysis of the cell proteome. The assembled EST sequence of the novel protein is confirmed by a peptide mass fingerprinting and RACE. The coding region of hcc-1 cDNA has 630 bases, which code for the 210 amino acids of the full-length protein. The unique DNA sequence at the 3′ untranslated region (218 bp) has been used to localize the gene to chromosome 7q22.1. A total of 690 bp at the 5′ untranslated region of hcc-1 has been identified and promoter activity has been demonstrated at this region. A number of uORFs, which is a common feature in proto-oncogenes and growth factors, are noted at the 5′ untranslated region.

The protein HCC-1 is localized to the nucleus region of two liver cell lines by immunofluorescence staining. Bioinformatics predictions show that the first 42 amino acids of the protein have identity matches to heterogenous nuclear ribonucleoproteins from various vertebrate species including human. The domain is also a putative bi-helical DNA-binding motif. The rest of the HCC-1 amino acid sequence has no known homology in vertebrates.

The cDNA of the hcc-1 is detected in tissue from various human organs. However, a marked increase in hcc-1 cDNA level is observed in pancreatic adenocarcinoma. An increase in hcc-1 cDNA level is also observed in well-differentiated hepatocellular carcinoma and its level decreases as the carcinoma progressed to a poorly differentiated stage. The increase in hcc-1 levels in both types of tumor are expected due to the same developmental origin of the two organs.

HCC-1 is proposed to be involved in nucleic acid binding and transcriptional control, and hence is involved in cell regulation. The protein and corresponding genetic sequence has therapeutic and diagnostic applications.

One aspect of the present invention is directed to an isolated nucleic acid molecule comprising a sequence of nucleotides, the expression of which, is differential or preferential in human hepatocellular carcinoma tissue or tissue from a related cancer relative to other tissue in said subject and/or in subjects not diagnosed with this condition.

Another aspect of the present invention provides an isolated peptide, polypeptide or protein or a derivative, homologue or analogue thereof which protein is differentially or preferentially produced in or by human hepatocellular carcinoma tissue or tissue from a related cancer relative to other tissue in said subject and/or in subjects not diagnosed with this condition.

Yet another aspect of the present invention is directed to a modulator of expression of a nucleic acid molecule which nucleic acid molecule is differentially or preferentially expressed in human hepatocellular carcinoma tissue or tissue from a related cancer relative to other tissue in said subject and/or in subjects not diagnosed with this condition.

Still another aspect of the present invention is directed to the use of a nucleic acid molecule, the expression of which is differential or preferential in human hepatocellular carcinoma tissue or tissue from a related cancer relative to other tissue in said subject an/or in subjects not diagnosed with having this condition in the manufacture of a medicament for the treatment of hepatocellular carcinoma or a related condition.

Another aspect of the present invention contemplates a method for diagnosing human hepatocellular carcinoma or a related condition in a subject or a propensity for said subject to develop human hepatocellular carcinoma or a related condition, said method comprising identifying expression of a gene which is differentially or preferentially expressed in tissue from subjects with hepatocellular carcinoma or a related condition relative to other tissue in said subject and/or subjects not diagnosed with this condition.

### BRIEF DESCRIPTION OF THE DRAWINGS

FIG. 1 is a representation of the nucleotide sequence of hcc-1 (SEQ ID NO:1).

FIG. 2 is a representation of the amino acid sequence of HCC-1 (SEQ ID NO:2); underlined sequences are amino acid sequences obtained by MS/MS analysis.

FIG. 3 is a representation of the nucleotide sequence of hcc-1 following amplification through long distance polymerase chain reaction (PCR) (SEQ ID NO:3) and used to construct an expression vector (873 bp).

FIG. 4 is a photographic representation showing PCR amplification of hcc-1 cDNA in normal and tumor liver tissues. M: DNA size marker; 1, Tumor tissue; 2, Normal tissue; 3, Negative control.

FIG. 5 is a representation of the untranslated region of hcc-1 (SEQ ID NO:4). Underlined sequences are the minicstrones or uORFs before the start of the P151 coding region with the start and stop codons in bold.

### DETAILED DESCRIPTION OF THE INVENTION

The present invention is predicated in part on the identification of gene expression products substantially present in or produced by tissue in subjects diagnosed with hepatocellular carcinoma or a related condition but substantially absent or in a substantially reduced amount in other tissues in the subject or in subjects not diagnosed with this condition.

Accordingly, one aspect of the present invention is directed to an isolated nucleic acid molecule comprising a sequence of nucleotides, the expression of which, is differential or preferential in human hepatocellular carcinoma tissue or tissue from a related condition relative other tissue in said subject and/or in to subjects not diagnosed with this condition.

Reference herein to an “expression product” includes reference to mRNA transcribed from a nucleotide sequence of a gene and/or an amino acid sequence, generally in the form of a peptide, polypeptide or protein, translated from the mRNA molecule. Expression products may be identified directly or indirectly such as via a complex (e.g. tRNA-amino acid complex) or via an effect. Terms such as “expression” or “expressed” means the expression of a gene sequence to produce an expression product.

The term “gene” is used in its broadest sense and includes cDNA corresponding to the exons of a gene. Accordingly, reference herein to a “gene” is to be taken to include: a classical genomic gene consisting of transcriptional and/or translational regulatory sequences and/or a coding region and/or non-translated sequences (i.e. introns, 5′- and 3′-untranslated sequences); or (ii) mRNA or cDNA corresponding to the coding regions (i.e. exons) and 5′- and 3′-untranslated sequences of the gene.

The term “gene” is also used to describe synthetic or fusion molecules encoding all or part of an expression product. In particular embodiments, the term “nucleic acid molecule” and “gene” may be used interchangeably.

The term “differential” or a related term such as “differentially” in relation to gene expression means that a gene sequence is expressed in one type of cell or tissue (e.g. cancerous cell or tissue) but is substantially not expressed in another cell or tissue. The term “preferential” or a related term such as “preferentially” in relation to gene expression means that a gene sequence is expressed at a higher level in one type of cell or tissue (e.g. cancerous cell or tissue) relative to another type of cell or tissue. The difference in expression levels may, for example, be from two-fold to 100-fold or from three-fold to 50-fold. In one embodiment, the gene is liver tissue of patients within hepatocellular carcinoma and is substantially not expressed in the normal liver.

Reference herein to a “subject” generally means a human subject although the present invention extends to other mammals which are capable of developing a homologous condition to human hepatocellular carcinoma. Such other mammals include livestock animals, laboratory test animals and companion animals.

The disease condition “hepatocellular carcinoma” also includes conditions related to hepatocellular carcinoma such as at the genetic, immunological, biochemistry, physiological, or aetiological levels. The terms “carcinoma”, “sarcoma” and “tumor” may be used interchangeably.

The term “isolated” in relation to a nucleic acid molecule or an expression product such as mRNA or a peptide, polypeptide or protein means that the nucleic acid molecule or expression product has undergone at least one purification step away from background material. Such a purification step includes gel electrophoresis, centrifugation, precipitation, chromatography such as HPLC or mass spectrometry such as MALDI-TOF MS.

A “nucleic acid molecule” may be RNA (e.g. mRNA) or DNA (e.g. genomic DNA or cDNA) or an RNA/DNA hybrid. A nucleic acid molecule may also be a gene as defined above. In one embodiment, the nucleic acid is in a vector such as an expression vector. In other embodiments, the nucleic acid is in single or double stranded, linear or covalently closed circular form. The present invention further extends to primers, probes, sense and antisense molecules, and ribozymes to be subject nucleic acid molecule.

The present invention further extends to the promoter region of the gene or functional variants of the promoter. The promoter may also be targeted in a therapeutic programme to modulate expression of the gene. Furthermore, the present invention extends to regulatory regions of the hcc-1 including 3′ and 5′ untranslated regions of the gene. Such regions may be used to genetically modulate expression of the gene.

In a particularly preferred embodiment, the promoter region of the hcc-1 is defined by the nucleotide sequence set forth in SEQ ID NO:4. The present invention extends to nucleotide sequence having at least 60% similarity to the nucleotide sequence set forth in SEQ ID NO:4 as well as a nucleotide sequence capable of hybridizing to the nucleotide sequence set forth in SEQ ID NO:4 or its complementary form.

The present invention further extends to expression products in isolated form. Preferably, the expression product is in the form of a peptide, polypeptide, or protein.

Another aspect of the present invention provides an isolated peptide, polypeptide, or protein, or a derivative, homologue, or analogue thereof, which protein is differentially or preferentially produced in or by human hepatocellular carcinoma tissue or tissue from a related cancer relative to other tissue in said subject and/or in subjects not diagnosed with this condition.

A “derivative” includes a single or multiple amino acid substitution, addition and/or deletion to the amino acid sequence normally associated with the peptide, polypeptide, or protein. Accordingly, a “derivative” includes a part, portion, or fragment of the peptide, polypeptide, or protein.

Conveniently, the part, portion, or fragment of the peptide, polypeptide, or protein contains antigenic determinants such that the part, portion or fragment is capable of interacting with antibodies to the expression product or to immune cells (e.g. T cells) sensitized to the expression product. A derivative also includes polymorphic variants or glycosylation variants as well as any alterations to molecules associated with the expression product such as lipids, carbohydrates, DNA or RNA, or other proteins.

Amino acid insertional derivatives of the peptide, polypeptide, or protein of this aspect of the present invention include amino and/or carboxyl terminal fusions as well as intra-sequence insertions of single or multiple amino acids. Insertional amino acid sequence variants are those in which one or more amino acid residues are introduced into a predetermined site in the molecule although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place.

Where the peptide, polypeptide or protein is derivatized by amino acid substitution, the amino acids are generally replaced by other amino acids having like properties, such as hydrophobicity, hydrophilicity, electronegativity, bulky side chains, and the like. Amino acid substitutions are typically of single residues. Amino acid insertions will usually be in the order of about 1-10 amino acid residues and deletions will range from about 1-20 residues. Preferably, deletions or insertions are made in adjacent pairs, i.e. a deletion of two residues or insertion of two residues.

Analogues including mimetics include molecules which contain non-naturally occurring amino acids as well as molecules which do not contain amino acids but nevertheless behave functionally the same as the peptide, polypeptide or protein. Analogues of the subject molecules contemplated herein include modifications to side chains, incorporation of unnatural amino acids and/or their derivatives during peptide synthesis and the use of crosslinkers and other methods which impose conformational constraints on the peptide molecule or their analogues.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of potential non-natural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | | Chexa L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenylethylamino) | Nmbc | | |
| cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

All these types of modifications may be important to stabilize the subject expression product. This may be important if used, for example, in the manufacture of a vaccine or therapeutic composition or agents for use in detection assays.

The present invention further contemplates chemical equivalents of the subject peptides, polypeptides and proteins. Chemical equivalents may not necessarily be derived from the subject molecule itself but may share certain conformational or functional similarities. Alternatively, chemical equivalents may be specifically designed to mimic certain physiochemical properties of the molecules. Chemical equivalents may be chemically synthesized or may be detected following, for example, natural product screening. Preferably, a chemical equivalent is a functional equivalent.

The amino acid variants referred to above may readily be made using peptide synthetic techniques well known in the art, such as solid phase peptide synthesis and the like, or by recombinant DNA manipulations. Techniques for making substitution mutations at predetermined sites in DNA having known or partially known sequence are well known and include, for example, M13 mutagenesis. The manipulation of DNA sequence to produce variant proteins which manifest as substitutional, insertional or deletional variants are conveniently described, for example, in Sambrook et al. (1989).

A “homologue” as referred to herein includes an expression product having a similar structure, function, genetic origin or immunogenic profile and which may be present in the same or a different cell type or in a different species of mammal.

In accordance with the present invention, it is proposed that the expression of a gene differentially or preferentially in hepatocellular carcinoma or a related condition provides a means for development of a range of therapeutic and diagnostic agents. In one particular case, the gene is associated with development, maintenance and/or growth of hepatocellular carcinoma or related condition. By targeting the gene, the expression of the gene and/or its expression product, it is proposed herein that this will reduce or inhibit development, growth or maintenance of the carcinoma and/or further facilitate another form of treatment conducted simultaneously or sequentially with.

Yet another aspect of the present invention is directed to a modulator of expression of a nucleic acid molecule which nucleic acid molecule is differentially or preferentially expressed in human hepatocellular carcinoma tissue or tissue from a related cancer relative to other tissue in said subject and/or in subjects not diagnosed with this condition.

In a related embodiment, there is provided a modulator of an expression product of a nucleic acid molecule which nucleic acid molecule is preferentially or differentially expressed in human hepatocellular carcinoma relative to subjects not diagnosed with this condition. A “modulator” may be an antagonist or agonist. In a preferred embodiment, the modulator is an antagonist.

The antagonist may be an antisense molecule or sense molecule (i.e. for co-suppression), a ribozyme, a DNA or RNA binding molecule (e.g. peptide, polypeptide or protein) which prevents or reduces expression of the target gene, an antibody or other molecule capable of interacting with the expression product. The antagonist may alternately reduce promoter activity and/or 5′ and/or 3′ untranslated regulatory regions.

One particularly useful group of antagonists are those identified following natural product screening or bioprospecting of sources such as a coral, plants, terrestrial environments, aquatic environments, micro-organisms and higher organisms.

The present invention further contemplates a composition such as a pharmaceutical composition comprising the modulator (eg. antagonist) and one or more pharmaceutically acceptable carriers and/or diluents.

Pharmaceutically acceptable carriers and/or diluents include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

The pharmaceutical composition may also comprise genetic molecules such as a vector capable of transfecting target cells where the vector carries a nucleic acid molecule capable of modulating expression of a nucleic acid molecule encoding binding partner. The vector may, for example, be a viral vector. In this regard, a range of gene therapies are contemplated by the present invention including isolating certain cells, genetically manipulating and returning the cell to the same subject or to a genetically related or similar subject.

Accordingly, the present invention provides a method of treating hepatocellular carcinoma, or a related condition, said method comprising administering to a subject in need of such treatment an antagonist of a gene or gene product which is differentially or preferentially expressed in tissue from subjects with hepatocellular carcinoma or a related condition relative to other tissue in said subject and/or subjects not diagnosed with this condition.

The present invention further provides for a method for identifying hepatocellular carcinoma or a related condition in a subject or a predisposition in a subject for developing such a condition. This aspect of the present invention is predicated in part on the identification of the expression product which is indicative of hepatocellular carcinoma or a predisposition for the development of same.

Still yet another aspect of the present invention is directed to the use of a nucleic acid molecule, the expression of which is differential or preferential in human hepatocellular carcinoma tissue or tissue from a related cancer relative to other tissue in said subject an/or in subjects not diagnosed with having this condition in the manufacture of a medicament for the treatment of hepatocellular carcinoma or a related condition.

The “expression product” may be identified by any number of means including the use of antibodies and probes designed to identify mRNA transcripts. Accordingly, another aspect of the present invention is directed to immunointeractive molecules such as antibodies to the expression product and their use in the development of diagnostic assays.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Douillard and Hoffman, 1981; Kohler and Milstein, 1975; 1976).

A wide range of immunoassay techniques are available as can be seen by reference to U.S. Pat. Nos. 4,016,043, 4,424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitative by comparing with a control ample containing known amounts of hapten.

Variations on the forward assay include a simultaneous assay, in which both sample and labelled antibody are added simultaneously to the bound antibody. These techniques are well known to those skilled in the art, including any minor variations as will be readily apparent. In accordance with the present invention, the sample is one which might contain an expression product such as a peptide, polypeptide or protein including mammalian cell extract, tissue biopsy, culture supernatant fluid or microbial or other cell extract. The sample is, therefore, generally a biological sample comprising biological fluid, and, as stated above, also extends to fermentation fluid and supernatant fluid such as from a cell culture.

In a typical forward sandwich assay, a first antibody having specificity for the expression product or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay.

The binding processes are well-known in the art and generally consist of cross-linking covalently binding or physically adsorbing. The polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 25° C. or above) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody.

Alternatively, a second labelled antibody, specific to the first antibody is exposed to the target-first antibody complex to form a target-first antibody-second antibody tertiary complex. The complex is detected by the signal emitted by the reporter molecule.

By “reporter molecule”, as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above.

In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength, the fluorescence observed indicates the presence of the hapten of interest. Immunofluorescene and EIA techniques are both very well established in the art and are particularly preferred for the present method. However, other reporter molecules, such as radioisotope, chemiluminescent, or bioluminescent molecules, may also be employed.

As stated above, when the expression product is mRNA, nucleic acid probes may be employed to detect the presence of the mRNA transcripts. A Northern blot is one example of detecting the presence of the transcripts. PCR and solid phase detection systems may also be used.

The detection of the expression product according to the present invention is conveniently provided in kit form with compartments adapted to contain the reagents for conducting the assay. Such reagents include antibodies, nucleic acid probes, PCR primers, enzymes, and/or diluents amongst other compounds.

The present invention further provides for the use of a nucleic acid molecule the expression of which is differential or preferential in human hepatocellular carcinoma tissue relative to other tissue or tissue from subjects not diagnosed with having this condition in the manufacture of a medicament for the treatment of hepatocellular carcinoma or a related condition.

The present invention is described hereinafter with reference to the detection of one particular gene designated hcc-1 from the human hepatocellular carcinoma cell line, HCC-M. The nucleotide sequence of hcc-1 is provided in SEQ ID NO:1. The corresponding expression product is a protein designated HCC-1 and this comprises an amino acid as set forth in SEQ ID NO:2. A PCR extended form for use in a vector is shown in SEQ ID NO:3.

Reference herein to “hcc-1” includes reference to its derivatives and homologues, “derivative” and “homologue” being as hereinbefore defined. Likewise, reference herein to the “HCC-1” polypeptide includes reference to all derivatives, homologues, and analogues thereof.

Another aspect of the present invention provides an isolated nucleic acid molecule comprising a sequence of nucleotides substantially as set forth in SEQ ID NO:1 or a sequence having at least 60% similarity thereto after optimal alignment or a sequence capable of hybridizing to SEQ ID NO:1 or its complementary form under low stringency conditions and wherein the expression of said nucleotide sequence is differential or preferential in human hepatocellular carcinoma tissue relative to other tissue or tissue from subjects not diagnosed with this condition or a derivative or homologue of said nucleic acid molecule, “derivative” and “homologue” being as hereinbefore defined.

Another aspect of the present invention provides an isolated polypeptide comprising an amino acid sequence substantially as set forth in SEQ ID NO:2 or an amino acid sequence having at least 60% similarity thereto or an amino acid sequence encoded by SEQ ID NO:1 or a nucleotide sequence having at least 60% similarity to SEQ ID NO:1 after optimal alignment or a nucleotide sequence capable of hybridizing to SEQ ID NO:1 under low stringency conditions or a derivative, homologue or analogue of the polypeptide.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length.

Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (1997). A detailed discussion of sequence analysis can be found in Unit 19.3 of Ausubel et al. (1998).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, 1962). However, the Tₘ of a duplex DNA decreases by 1° C. with every increase of 1% in the number of mismatch base pairs (Bonner and Laskey, 1974). Formamide is optional in these hybridization conditions.

Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The present invention further extends in a modified nucleotide sequence encoding HCC-1 where a nucleotide sequence is optimized to facilitate greater expression in a particular host cell. Accordingly, the present invention contemplates a method for the construction of a nucleic acid molecule comprising a non-naturally occurring nucleotide sequence, said method comprising constructing in a particular reading frame, a contiguous sequence of codons which encode a sequence of amino acids of a polypeptide where one or more codons are selected to express at a higher level in a particular host cell or in vitro expression system relative to the corresponding codons in the naturally occurring nucleotide sequence encoding the same polypeptide, wherein the selected codons are preferably used by a host cell, and wherein the codon for Phe may be selected from the group comprising UUU and UUC, the codon for Ser may be selected from the group comprising UCU, UCC, UCA, UCG, AGU and AGC, the codon for Tyr may be selected from the group comprising UAU and UAC, the codon for Cys may be selected from the group comprising UGU and UGC, the codon for Trp may be selected from the group comprising UGG, the codon for Leu may be selected from the group comprising CUU, CUC, CUA, CUG, UUA and UUG, the codon for Pro may be selected from the group comprising CCU, CCC, CCA and CCG, the codon for His may be selected from the group comprising CAU and CAC, the codon for Gln may be selected from the group comprising CAA and CAG, the codon for Arg may be selected from the group comprising CGU, CGC, CGA, CGG, AGA and AGG, the codon for Ile may be selected from the group comprising AUU, AUC and AUA, the codon for Met may be selected from the group comprising AUG and GUG, the codon for Thr may be selected from the group comprising ACU, ACC, ACA, and ACG, the codon for Asn may be selected from the group comprising AAU and AAC, the codon for Lys may be selected from the group comprising AAA and AAG, the codon for Val may be selected from the group comprising GUU and GUC, the codon for Ala may be selected from the group comprising GUA, GUG, GCU, and GCC, the codon for Asp may be selected from the group comprising GCA, GCG, GUA and GAC, the codon for Glu may be selected from the group comprising GAA and GAG, and the codon for Gly may be selected from the group comprising GGU, GGC, GGA, and GGG.

Reference herein to a “host cell” refers to a cell or cells derived such as from a group including but not limited bacteria, yeasts, fungi, plants, insects and animals. A host cell is capable of expressing a peptide, polypeptide or protein from a nucleic acid molecule. The term “host cell” may also be read as a “foreign” cell meaning that the host cell is not from the species or strain of organism from which a particular coding sequence or non-coding sequence is derived. The host cell may however be a genetically modified form of the original source organism. In the case of a coding sequence or non-coding sequence derived from P. gingivalis or a related organism, the suitable host cell for expression of a modified sequence includes E. coli stains such as but not limited to, WA803, WA802, RR1, Q359, Q538, P2392, NM621, NM554, NM477, MC4100, MC1061, DL538, DB1316, CSH18, CES200, C600hfi, C600, BNN₁O₂, BNN93, BL21(DE3), and BHB2690.

Other suitable bacterial host cells include but are not limited to the following bacteria, Aminobacterium mobile DSM 12262, Aminomonas paucivorans DSM 12260, Asaia bogorensis JCM 10569, Bacteroides thetaiotaomicron BTX, Burkholderia kururiensis JCM 10599, Desulfovibrio dechloracetivorans SF3, Escherichia coli HS(pFamp)R, Kocuria rhizophila DSM 11926, Methylobacterium mesophilicum AM24, Mycobacterium avium MAC 511, Mycobacterium avium MAC 101, Phormidium corium, Pseudomonas aeruginosa ERC 1, Pseudomonas aeruginosa HER-1001, Pseudomonas aeruginosa HER-1002, Pseudomonas aeruginosa HER-1010, Pseudomonas aeruginosa HER-1009, Pseudomonas aeruginosa HER-1016, Pseudomonas aeruginosa HER-1017, Pseudoxanthomonas broegbernensis DSM 12573, Ralstonia gilardii LMG 5886, Shewanella frigidimarina ACAM 591, Shewanella gelidimarina ACAM 456, Streptococcus pneumoniae MS22, Streptococcus pneumoniae Fi10, Streptococcus pneumoniae 51702, Streptococcus pneumoniae TW31, Streptococcus pneumoniae TW17, Thiomicrospira frisia JB-A2, Thiomicrospira kuenenii JB-A 1, Treponema lecithinolyticum OMZ 685, Treponema maltophilum BR, Treponema maltophilum PNA1, Treponema maltophilum HO2A, and Ureaplasma urealyticum.

Still other suitable host cells include but are not limited to the following fungal cells Hyphodontia australis 231, Kluyveromyces lactis CK56-7A, Kluyveromyces lactis CW64-1C, Prosthemium asterosporum A1, Prosthemium betulinum B1, Saccharomyces cerevisiae 1A-H 19 [psi-], Saccharomyces cerevisiae 5V-H 19 [psi-], Saccharomyces cerevisiae 1-5V-H 19, Saccharomyces cerevisiae PS-5V-H 19, Saccharomyces cerevisiae C10B-H49, Saccharomyces cerevisiae 9V-H70 [PIN+], Saccharomyces cerevisiae 4V-H73, Saccharomyces cerevisiae 17G-H73, Saccharomyces cerevisiae 3B-H72, Saccharomyces cerevisiae DL1, Saccharomyces cerevisiae GW226, Saccharomyces cerevisiae JM43-GD7, Saccharomyces cerevisiae MCC318, Saccharomyces cerevisiae NB39-5D, Saccharomyces cerevisiae NGB108, Saccharomyces cerevisiae PTH43, Saccharomyces cerevisiae PTH352, Saccharomyces cerevisiae PTY11, Saccharomyces cerevisiae TF112, Saccharomyces cerevisiae TWM10-41, Saccharomyces kluyveri GRYL 175, Saccharomyces kluyveri MCC328, and Saccharomyces kluyveri NB 180.

Suitable mammalian host cells for expression include, the mammalian cell lines including but not limited to mammalian cell line, 22Rvl Human prostate carcinoma, A7 Human melanoma, B13-24 Chinese hamster, antibody producing, EOC 2 Mouse microglia; macrophage, EOC 13.31 Mouse microglia; macrophage, EOC 20 Mouse microglia; macrophage, HAAE-2 Human normal abdominal aorta, HS-5 Human HPV-16 E6/E7 transformed, I-11.15 Mouse macrophage, I-13.35 Mouse macrophage, KMA Human macrophage; monocyte, NCI-BL1770 Human Epstein-Barr transformed B lymphoblastoid line, NCI-BL2107 Human Epstein-Barr transformed B lymphoblastoid line, NCI-BL2141 Human Epstein-Barr transformed B lymphoblastoid line, NCI-H211 Human carcinoma; small cell lung cancer, NCI-H841 Human carcinoma; variant small cell lung cancer, NCI-H847 Human carcinoma; classic small cell lung cancer, NCI-H1341 Human carcinoma; small cell lung cancer, NCI-H2122 Human adenocarcinoma; non-small cell lung cancer, RTgill-W1 Rainbow trout, normal gill, F-1.CN5a.1 Human erythroleukemia, TK# 1 Mouse disrupted interferon regulatory factor 2 (IRF-2) gene, and TOV-112D Human primary malignant adenocarcinoma

Reference herein to a nucleic acid molecule or nucleotide sequence being “non-naturally occurring” or other wise “non-natural” is meant to be considered in its broadest sense to include a nucleic acid molecule or nucleic acid sequence which has been artificially created by a chemical synthetic or recombinant means or by directed or controlled genetic processes including homologous recombination. The selection of a particularly preferred codon or nucleotide sequence is deemed here to be an example of rendering the resulting nucleic acid molecule or nucleotide sequence as non-naturally occurring.

Reference herein to an “in vitro expression system” includes an in vitro translation system and refers to a cytoplasmic or cell extract comprising molecules such that when the cell extract is provided with a nucleic acid sequence that encodes a peptide polypeptide or protein, the cell extract is competent to express the peptide polypeptide or protein. Such extracts may be produced from cells or tissues derived such as from but not limited to the group including bacteria, yeasts, fungi, plants, insects, and animals.

The hcc-1 nucleic acid molecule may be resident in isolated form as a linear, single or double stranded molecule or it may be resident in a vector such as an expression vector.

The present invention further provides transgenic cells carrying hcc-1 or otherwise producing HCC-1. Such cells include bacteria, yeast, insect, animal, and mammalian cells.

Yet another aspect of the present invention provides an antisense molecule to hcc-1 transcript whereby the antisense molecule reduces expression of hcc-1 by from about 5% to about 100% or from about 10% to about 80% or from about 20% to about 70% relative to a control.

The hcc-1 gene is expressed in hepatocellular carcinoma tissue but is substantially not expressed in other tissue. The gene and its expression product, HCC-1, provide a convenient marker for the cancer condition and/or for the development of antagonists of hcc-1 expression or HCC-1 activity.

It is proposed that HCC-1 is involved in nucleic acid binding and transcriptional control. Modulating expression of hcc-1 or modulating HCC-1 activity provides a means of modulating cell regulation. Accordingly, another aspect of the present invention contemplates a method of modulating one or more activities within a cell, said method comprising modulating expression of hcc-1 gene expression or the activity of HCC-1 for a time and under conditions sufficient to modulate the cell activity.

Reference to cell activity includes at least one physiological, biochemical, immunological, or other biological property within the cell or on the cell surface. For example, in so far as HCC-1 is involved in transcription, increasing levels of HCC-1 or decreasing levels of this protein will effect the level of transcription of the target gene.

The present invention is further described by the following non-limiting Examples.

### EXAMPLE 1

### Culture Techniques

The HCC-M cell line was cultured in Dulbelcco's modified Eagle medium (DMEM) from Gibco BRL (Life Technologies, Gaithersburg, Md., USA) containing 10% v/v fetal calf serum (FCS) from Biological Industries (Haemek, Israel) at 37° C. in 5% CO₂/95% air at 95% relative humidity. The cells were harvested once a monolayer culture was attained. During harvesting, the cells were rinsed with DMEM without FCS. Cell detachment was performed by incubation with a solution of 0.5 g/L trypsin and 0.2 g/L ethylenediaminetetraacetic acid [EDTA] (Gibco BRL). After 15 mins, DMEM containing FCS was added to terminate the action of the protease. The resulting suspension was centrifuged at 2000 rpm for 5 mins at 4° C. After discarding the supernatant fluid, the cells were resuspended with DMEM without FCS and centrifuged at 10000 rpm for 5 mins at 4° C. After centrifugation, the supernatant was removed and the cell pellet stored at −80° C. until further use.

### EXAMPLE 2

### Sample Preparation

Harvested HCC-M cells were disrupted with a cocktail of 7 M urea (Bio-Rad Laboratories, Hercules, Calif., USA). 2 M thiourea (Fluke Chemie AG, Buchs, Switzerland), 4% v/v 3-[(3-cholamidopropyl)dimethylammonio]-1-propanesulphonate (CHAPS) (USB, Amersham Pharmacia Biotech AB, Uppsala, Sweden), 40 mM tris(hydroxymethyl)aminomethane (Tris) (J. T. Baker, Phillipsburg, N.J., USA) and 1 mM phenylmethylsulphonyl fluoride (PMSF) (Sigma Chemical Co., St. Louis, Mo., USA). The resulting cell lysate was subjected to physical shearing by passing it through a syringe fitted with a 21 G needle, followed by syringes with 25 G and 27 G needles successively, and the addition of 50 μg/ml DNase I (from bovine pancreas, grade II, Boehringer Mannheim, GmbH, Mannheim, Germany) and 50 μg/ml RNase A (from bovine pancreas, Boehringer Mannheim). The sample was then centrifuged using a Beckman TL-100 Tabletop Ultracentrifuge (Palo Alto, Calif., USA) at 85000 rpm (297785×g) for 2 hrs at 15° C.

### EXAMPLE 3

### Two-Dimensional Gel Electrophoresis

The first dimensional IEF was performed on precast 18 cm IPG strips (Amersham Pharmacia Biotech) at 20□C with a maximum current setting of 50 μA/strip using an Amersham Pharmacia IPGphor IEF unit. The strips were rehydrated for a minimum of 10 hrs in ceramic strip holders in 350 FL of sample containing 7 M urea, 2 M thiourea, 4% v/v CHAPS, 1 mM PMSF, 20 mM dithiothreitol (DTT) (Bio-Rad) and 0.5% v/v IPG buffer (Amersham Pharmacia Biotech). The amount of protein loaded was ˜150 μg for analytical gels and ˜400 μg protein for preparative gels. A low voltage of 30 V was applied during rehydration. After rehydration, IEF run was carried out using the following conditions: (i) 500 V, 500 Vhr; (ii) 1,000 V, 1000 Vhr; and (iii) 8000 V, 32000 Vhr. Voltage increases were performed on a step-wise basis. Before carrying out the second-dimensional sodium dodecyl sulphate-polyacrylamide gel electrophoresis (SDS-PAGE), the strips were subjected to a two-step equilibration. The first was an equilibration buffer consisting of 6 M urea, 30% v/v glycerol (BDH Laboratory Supplies, Poole, England), 2% w/v SDS (Merck KGaA, Darmstadt, Germany), 50 mM Tris-HCl (pH 6.8) and 1% w/v DTT. The second step was with a buffer consisting of 6 M urea, 30% v/v glycerol, 2% w/v SDS, 50 mM Tris-HCl (pH 8.8) and 2.5% w/v iodoacetamide (IAA) (Sigma). After the IPG strips were transferred onto the second-dimension SDS-PAGE gel, the strips were sealed in place with 0.75% agarose (USB). SDS-PAGE was performed on 1.0 mm thick 10% and 10% w/v polyacrylamide gels at a constant voltage of 110 V at 10° C. using an Amersham Pharmacia Iso-Dalt electrophoresis unit.

### EXAMPLE 4

### Silver Staining

Silver staining of the gels was performed using published procedures with some modifications. The gels were fixed in 50% v/v methanol (Merck), 5% v/v acetic acid (Merck) in water for 30 mins followed by washing in 50% methanol in water for 10 mins. Then the gels were washed again with water for 60 mins and sensitized with 0.02% sodium thiosulphate (Merck) for 2 mins. After the gels were rinsed twice with water for 1 min each, they were incubated in chilled 0.1% w/v silver nitrate (Merck) for 40 mins at 4° C. After discarding the silver nitrate and rinsing with two changes of distilled water for 1 min each, the gels were developed in 0.04% v/v formalin (35% v/v formaldehyde in water) (Merck) in 2% w/v sodium carbonate (Merck). When the desired intensity was attained, the developer was discarded and the gel incubated with 1.46% w/v EDTA disodium dihydrate (Bio-Rad) for 10 mins to stop the development. The staining procedure was completed by three rinses with water for 5 mins each. Stained gels were scanned using a Molecular Dynamics Personal Densitometer SI (Sunnyvale, Calif., USA).

### EXAMPLE 5

### Image Analysis

The gels were analyzed by traditional eyeballing method and the PDQuest (Version 6.1) software from Bio-Rad Laboratories. Using the Spot Detection Wizard function, the scanned gels were processed to remove vertical and horizontal streaks and enhance the spots before crosshairs were placed on the detected spots.

### EXAMPLE 6

### Enzymatic Digestion of Protein Spots

Silver stained spots were excised manually with a homemade plastic plunger and transferred to a 96-well polypropylene microtiter plate. Each excised spot was washed with 175 μL of 25 mM Tris-HCl (pH 8.5) in 50% acetonitrile (Applied Biosystems, Foster City, Calif., USA). The plate was sealed with an adhesive film and stored at 4° C. for at least 24 hrs. This step was critical for the equilibration of gel spots as it allowed for more efficient enzyme digestion. Prior to the addition of trypsin, the washing solution was replaced with a fresh aliquot of solution and plates were incubated with shaking for 20 mins at 37° C. The washing solution was then aspirated and gel spots were dried in a Savant Automatic Environment SpeedVac AES2010 centrifugal concentrator (Holbrook, N.Y., USA) for 30 mins. Enzymatic digestion was performed with the addition of 10 ΦL of 0.02 μg/L trypsin (Promega Corporation, Madison, Wis., USA) in 25 mM ammonium bicarbonate (pH 8.5) (Sigma) to each gel piece and incubated at 37° C. overnight with shaking. To enhance peptide extraction, 10 μL of 0.1% trifluoroacetic acid (TFA) (Sigma) in 50% acetonitrile was added to each well and the microtiter plate sonicated for 10 mins in an ultrasonic water bath (Crest Ultrasonics, NJ, USA).

### EXAMPLE 7

### Matrix-Assisted Laser Desorption/Ionization—Time of Flight (MALDI-TOF)-MS Analysis of Tryptic Peptides

Mass analyses were performed according to previously published methods using a PerSeptive Biosystems Voyager-DE STR MALDI-TOF MS (Framingham, Mass., USA). In essence, 1 μL of the extracted sample from each of the microtitre wells was dispensed onto a MALDI sample plate along with 1 □L of matrix solution (10 mg/mL α-cyano-4-hydroxycinnamic acid (Sigma), 0.1% TFA, 50% acetonitrile). The samples were allowed to dry under ambient conditions. For each sample, the average of 256 spectra was acquired in the delayed extraction and reflector mode. The average of 4 scans (each containing 64 spectra) that passed the accepted criterion of peak intensity was automatically selected and saved. Spectra were automatically calibrated upon acquisition using a two-point calibration with residual porcine trypsin autolytic fragments (842.51 and 2210.10 [M+H⁺] ions). Assignment of peaks was done manually, measured peptide masses were excluded if their masses corresponded to trypsin autodigestion products or to identified proteins adjacent to the spot being analyzed.

### EXAMPLE 8

### Quadrupole-Time-Of-Flight (TOF) Tandem MS Analysis of Tryptic Peptides

De novo peptide sequencing was performed using a PE Sciex QSTARθ tandem mass spectrometry system (Concord, Ontario, Canada). The tryptic digested protein sample cleanup was conducted using the C₁₈ Zip Tip (Millipore) and eluted with 3 μl of 60% v/v methanol/5% v/v formic acid. One μl sample was loaded onto the spray needle for nanospray (Protana, DK) analysis. The spray was started by applying a spray potential of 800 volts. The spray lasted for about 25 mins for each sample. QSTAR was operated with resolution of about 10,000 FWHM. Data acquisition were done using TOF Tune software and data were processed using Biomultiview software. The “y” and “b” ions weightage were used to get the sequence from MS/MS of peptides.

### EXAMPLE 9

### Database Searching and Identification of Proteins

The proteins were identified by searching in SWISS-PROT and NCBI non-redundant databases using MS-Fit (Protein Prospector, UCSF, San Francisco, USA). All mass searches were performed using a mass window between 1000 and 10000 Da, and included human and mouse sequences. The search parameters allowed for oxidation of methionine, N-terminal acetylation, carboxyamidomethylation of cysteine and phosphorylation of serine, threonine and tyrosine. The criteria for positive identification of proteins were set as follows: (i) at least four matching peptide masses; (ii) 50 ppm or better mass accuracy; and (iii) identified proteins□ molecular weight and pI should match estimated values obtained from image analysis.

### EXAMPLE 10

### Identification of hcc-1

Proteins from complex cell lysates were obtained from tissue samples or cell lines and separated using two-dimensional SDS-PAGE. The separated proteins were then excised from the gel and subjected to an in-gel enzymatic digest. The resultant peptides were then analyzed using a MALDI-TOF MS and a Quadrupole-TOF Tandem MS. Database searches were performed with the mass spectrometric data obtained.

The present invention arose initially using the MS/MS data obtained from the Quadrupole-TOF Tandem MS. The sequences of four peptide fragments were identified by this method. These data were used to search the protein databases and no matches with any known proteins were found.

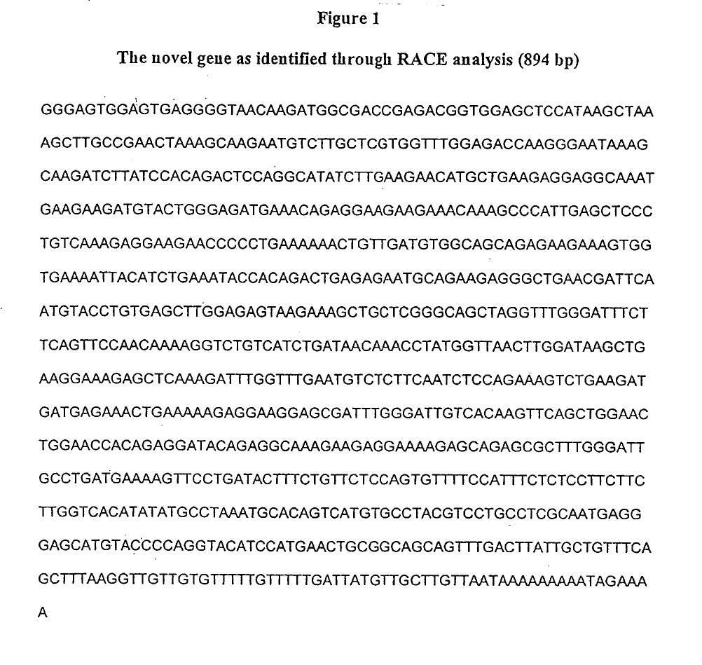

The HCC-M cells were grown to confluence and RNA of the cells were extracted through a standard guanidine isothiocyanate method (Chomczynski and Sacchi, 1987). Poly-A RNA was then purified from the RNA through poly-T resin binding. DNA primers were made based on the peptide sequences and a rapid amplification of cDNA ends (RACE) was performed on the poly-A RNA. The 5′-RACE and 3′-RACE results were compared and stitched together to give a full-length gene of 894 bases (FIG. 1; hcc-1; SEQ ID NO:1). The 3′-RACE product contained the poly-A tail of the gene.

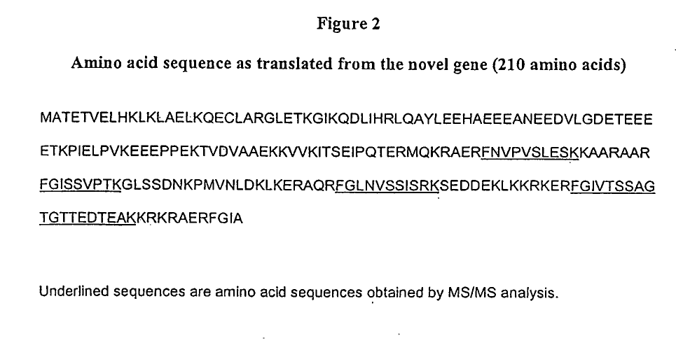

The open reading frame (ORF) of this novel gene was determined from the various possible ORFs to contain a protein of 210 amino acids in length (FIG. 2; HCC-1; SEQ ID NO:2). The novel protein has a theoretical pI and molecular weight of 6.1 and 23.6 kD, respectively. DNA primers at the extreme ends of this novel gene (the sense primer were situated before the start codon at the 5′ end and the anti-sense primer before the poly-A tail at the 3′ end) were synthesized and a long distant polymerase chain reaction (PCR) was then performed on the HCC-M poly-A RNA.

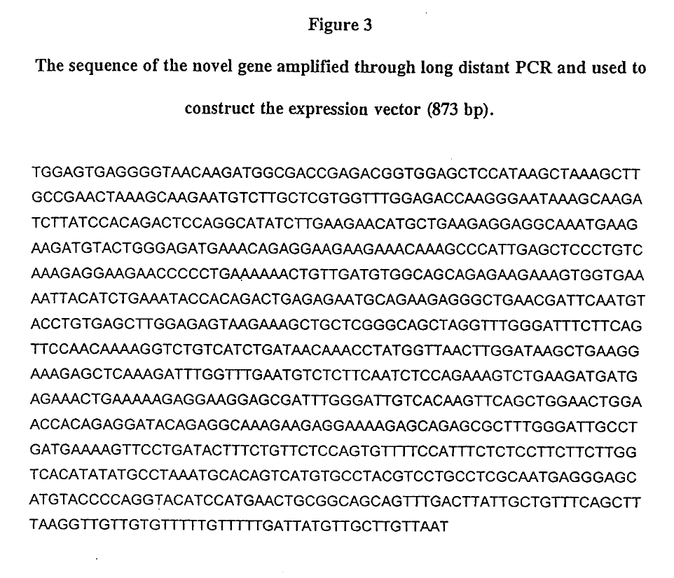

The product (873 bp, FIG. 3; SEQ ID NO:3) was TA cloned into pGEM-T (Promega, Inc., USA) and subsequently transferred to an expression vector pQE-30 (Qiagen, Germany). Host for the above two vectors was Escherichia coli strain DH5α. The expression product contained a 6×His tag at the amino end of the protein.

A multiple tissue panel containing 1st strand cDNA from both human normal and tumor tissues was obtained from Clontech (USA). Highly specific primers (Tₘ˜70□C) were generated based on the novel gene sequence and used to perform a PCR screening on the multiple tissue panel. Results are as shown in Table 2. Human healthy liver tissue (obtained during liver transplant operation) and a commercial human normal liver cDNA library (Gibco BRL, USA) were also found to express this gene at low abundance.

TABLE 2
Multiple tissue panel* screening for the novel gene

| Normal Tissue | Result | Tumor Tissue¹ | Result |
|---|---|---|---|
| Colon | − | Breast carcinoma (GI-101) | +/− |
| Ovary | − | Lung carcinoma (LX-1) | − |
| Peripheral blood | − | Colon adenocarcinoma (CX-1) | − |
| leukocyte | | | |
| Prostate | +/− | Lung (GI-117) | − |
| Small intestine | − | Prostatic adenocarcinoma | − |
| | | (PC3) | |
| Spleen | + | Colon adenocarcinoma | − |
| | | (GI-112) | |
| Testis | ++ | Ovarian carcinoma (GI-102) | − |
| Thymus | + | Pancreatic adenocarcinoma | +++ |
| | | (GI-103) | |
| Brain | − | | |
| Heart | + | | |
| Kidney | +/− | | |
| Liver | +/− | | |
| Lung | − | | |
| Pancreas | + | | |
| Placenta | − | | |

1st stand cDNA
Tumor tissues were propagated as xenograft in athymic nude mice.

### EXAMPLE 11

### Comparison of Expression of hcc-1 in Normal Versus Tumor Liver Tissues

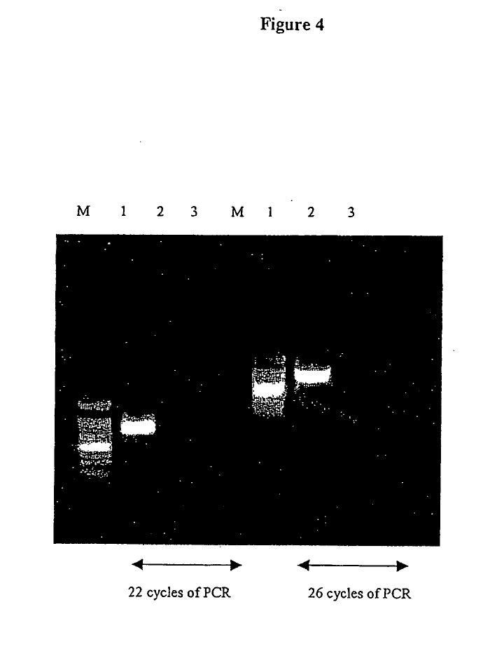

FIG. 4 shows that the hccl-1 gene is preferentially transcribed in the liver of a subject with hepatocellular carcinoma but is substantially not transcribed in the normal liver. In this experiment, an equal amount of cDNA from a normal liver tissue and from tissue of a subject diagnosed with hepatocellular carcinoma were amplified by PCR at high stringency. hcc-1 in tumor tissue is found to be about 10 to 50 times the amount in normal liver tissue (FIG. 4).

### EXAMPLE 12

### Chromosome Localization

The chromosomal location of the Hcc-1 was identified by radiation hybrid mapping of the human genome (Barrett 1997). Two human radiation hybrid-mapping panels were used for this purpose. The Genebridge4 panel is adopted by the European Consortium on Radiation Hybrid Mapping and is widely used in genome mapping projects, while the Stanford G3 panel is created at the Stanford Human Genome Centre for medium resolution chromosome localization of markers. Briefly, DNA from each of the 93 cell lines from Genebridge 4 and 83 cell lines from Stanford G3 were used as PCR template for primers designed from the 3′-untanslated region of Hcc-1. The results were scored for the presence and absence of a PCR product from Hcc-1. These data were then submitted to Whitehead/MIT RH server (for Genebridge 4) and Stanford Human Genome Center (for Stanford G3) where it was tested against the framework markers that have already been assayed. The placement of the gene was the best possible placement when scored against the framework markers at the time of experiment. Hcc-1 is assigned to chromosome 7 at position 7q22.1, 3.36 cR from marker D75651.

### EXAMPLE 13

### Sub-Cellular Localization

Antibody against Hcc-1 protein was raised in rabbits. Hcc-1 protein sub-cellular localization was performed on Huh7 and HCC-M cells by immunofluorescent staining. The cells were grown on glass cover slip, fixed with paraformaldehyde and detected with antibody against Hcc-1. Co-localization was performed with antibodies against mitochondria and golgi body (LabVision). The images from the individual antibody staining were scanned by confocal microscope and overlaid to form a composite image. Hcc-1 protein was localized to the nucleus.

### EXAMPLE 14

### Immunological Studies

Antibodies against cloned Hcc-1 protein was raised in rabbits. Its sensitivity and specificity were verified by Western blots detection of HCC-M lysate in 2D gel. However, Hcc-1 protein was not detectable in Western blots of 2D gel electrophoresis or 1D SDS-PAGE of human liver tissues. Hcc-1 cDNA expression levels in two paired (non-tumor and hepatocellular carcinoma) human liver tissues are as followed. Both subjects were positive for hepatitis B virus infection. Subject A had well differentiated tumor while subject B has poorly differentiated tumor.

TABLE 3
Expression of cDNA in Tumor
and Non-Tumor Tissue

| Subject | Liver Tissue | cDNA |
|---|---|---|
| A | Non-tumor | − |
| | Tumor | +++ |
| B | Non-tumor | ++ |
| | Tumor | + |

From the above studies, it can be seen that Hcc-1 is differentially expressed. Its cDNA levels were raised in pancreatic adenocarcinoma as compared to healthy pancreas (see Table 3). It is also increased in well-differentiated hepatocellular carcinoma and its level seemed to decrease as the tumor progressed to poorly differentiated hepatocellular carcinoma. The pancreas and liver have the same developmental origin (Bock et al. 1997) and Hcc-1 is increased in both types of tumor.

### EXAMPLE 15

### Promoter Study for P-151

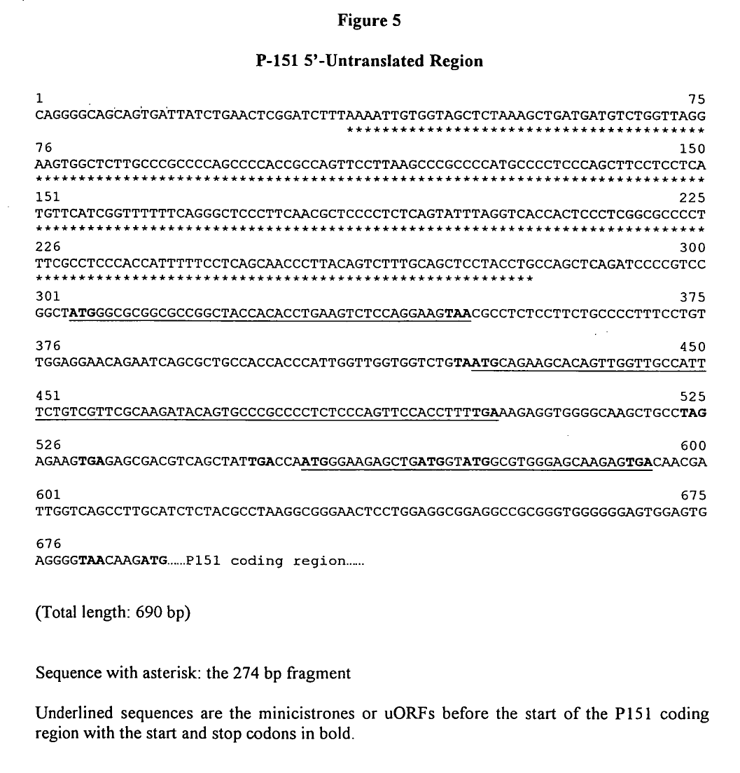

Four libraries of uncloned, adaptor-ligated high quality human genomic DNA fragments were obtained from Clontech, Inc (USA). Nested PCR was performed with primers derived from the adaptors and known Hcc-1 gene sequence at the 5′-untranslated region and exon 1 sequence. Two of the libraries were amplifiable (with DNA product of 690 bp and 3.8 kb respectively). The PCR products were TA cloned and sequenced. The DNA sequence for the 690 bp fragment is shown in FIG. 5. Multiple mini-cistrones were noted from nucleotide sequence 300 to 690 bp.

The 690 bp fragment was then ligated to a vector lacking eukaryotic promoter and enhancer sequences (pSEAP2 from Clontech, Inc). The vector contains a secreted human placental alkaline phosphatase gene (SEAP) downstream of the multiple cloning sites. The construct (5 μg) were transfected by a liposome-based transfection reagent (Clontech, Inc) into mammalian Huh7 cells. Normalization was performed by co-transformation with a vector containing the lacZ gene.

Promoter activity was determined by assaying for the secreted alkaline phosphatase activity 48 hours post-transfection using the fluorescent substrate 4-methylumbelliferyl phosphate (MUP). Low promoter activity was observed (10 ng SEAP expressed per 5 μg DNA). When the SV40 early promoter was added to the vector, increased SEAP transcription was observed (90 ng SEAP expressed). However, high transcription activity was obtained when the 690 bp fragment was constructed into a vector containing SV40 early enhancer sequence (190 ng SEAP expressed). This indicates that an enhancer element is needed for the transcriptional activity of the Hcc-1 promoter.

To bypass the mini-cistrones, 274 bp from the 5′ end of the 690 bp fragment was amplified and inserted into the pSEAP2 vector. No activity was observed when the pSEAP2 vector was constructed without SV40 early enhancer or promoter sequences. Transcriptional activity was observed at half (110 ng of SEAP expressed) of that from 690 bp fragment when the SV40 early enhancer sequence was included in the construct. The results showed that the promoter region is located primarily at the middle of the identified 5′-unstranslated region of the Hcc-1 gene. The enhancer sequence is probably further upstream from the 690 bp sequence.

Promoter region was predicted from 294 to 544 bp by ProScan (ver 1.7). This is in accordance with the promoter studies above where the 274 bp fragment at the 5′ end has less transcriptional activity compared to the complete 690 bp fragment.

The occurrence of a long 5′ untranslated region with mini-cistrones or upstream open reading frames (uORFs) is not uncommon. It is found in a number of proto-oncogenes and growth factors (Willis 1999). It is a structure used in transcriptional regulation and translational control (Brown & Schreiber 1996; Clemens & Bommer 1999) of genes whose products are important for cell growth.

### EXAMPLE 16

### Bioinformatics Findings on HCC-1

The Conserved Domain Database (CDD) with Reverse Position Specific BLAST search on the 1-42 amino acids of HCC-1 gave the result as a SAP domain (e-value of 5e-04), which is a putative bi-helical DNA-binding motif predicted to be involved in chromosomal organization and transcriptional regulations (Massari & Murre 2000) found in diverse nuclear proteins. This is supported by PredictProtein where amino acid sequence 197-203 was predicted to contain the nuclear localization signal. There is no predicted trans-membrane segment (using TMAP and PredictProtein), no mitochondrial targeting sequence (PSORT), and no secretory signal (SignalP).

Using PSI-BLAST on non-redundant database, amino acid sequence 1-42 of HCC-1 was matched to vertebrate heterogenous nuclear ribonucleoprotein with identities match of above 45%:
Heterogenous nuclear ribonucleoprotein U (AF073992) of Mus musculus
[Expect=0.005, Identities=21/42 (50%), Positives=29/42 (69%)]
SP120 (D14048) (nuclear scaffold protein that binds the matrix attachment region DNA) of Rattus norvegicus
[Expect=0.005, Identities=21/42 (50%), Positives=29/42 (69%)]
ROU_HUMAN Heterogenous nuclear ribonucleoprotein U (HNRNP U) (Scaffold Attachment Factor A) (SAF-A) (Q00839) of Homo sapiens
[Expect=0.012 Identities=20/42 (47%), Positives=29/42 (68%)]
hnRNP U protein (X65488) of Homo sapiens
[Expect=0.012, Identities=20/42 (47%), Positives=29/42 (68%)]
Scaffold attachment factor A (AF068847) of Xenopus laevis
[Expect=0.021, Identities=20/37 (54%), Positives=26/37 (70%)]

Using FASTA3 on SWALL non-redundant database, HCC-1 was matched to various invertebrate translated proteins with E-value below 0.03:
Q9VHC8 CG8149 protein of Drosophila melanogaster
[Expect=8e-06]
Q9N3GO Hypothetical protein Y53G8AR.d of Caenorhabditis elegans
[Expect=0.0005]
Q9LZ08 Hypothetical 22.8 KDA protein of Arabidopsis thaliana
[Expect=0.021]
074871 Conserved hypothetical protein of Schizosaccharomyces pombe (Fission yeast)
[Expect=0.024]

Physically, this HCC-1 protein may have 2 to 3 domains from coiled-coil and low complexity region predictions:
PredictProtein Coiled-Coil prediction—the coil is most probably at 30-51 positions. The next possible coiled-coil is at 146-160 positions. Coiled-coil most probably separates the different domains.
COILS ver 2.2 (Lupas)—at aa 25-64 and aa 145-172.
SEG Low Complexity regions predicted 2 regions: at aa 42-79 and aa 165-179.

It is to be understood that the foregoing description and specific embodiments shown herein are merely illustrative of the invention and its principles. Modifications and additions to the invention may readily be made by those skilled in the art without departing from the spirit and scope of this invention.

The articles in scientific periodicals and any patent literature cited hereinabove are hereby expressly incorporated by reference in their entireties for all purposes.

### BIBLIOGRAPHY

Altschul et al., Nucl. Acids Res. 25: 3389. 1997.
Ausubel et al., “Current Protocols in Molecular Biology” John Wiley & Sons Inc, 1994-1998, Chapter 15.
Barrett J H 1992. Genetic mapping based on radiation hybrid data. Genomics. 13: 95-103.
Bock P, Abdel-Moneim M, Egerbacher M. Development of Pancreas. 1997. Microscopy Research & Technique. 37: 374-383.
Bonner and Laskey, Eur. J. Biochem. 46: 83, 1974.
Brown E J, Schreiber S L. 1996. A signaling pathway to translational control. Cell. 86: 517-520.
Chomczynski, P. and Sacchi, N., Anal. Biochem. 162:156169
Clemens M J, Bommer U-A. 1999. Translational control: the cancer connection. The International Journal of Biochemistry and Cell Biology. 31: 1-23.
Douillard and Hoffman, Basic Facts about Hybridomas, in Compendium of Immunology Vol. II, ed. by Schwartz, 1981
Kohler and Milstein, Nature 256:495-499, 1975
Kohler and Milstein, European Journal of Immunology 6:511-519, 1976.
Marmur and Doty, J. Mol. Biol. 5: 109, 1962.
Massari M E, Murre C. 2000. Helix-loop-helix proteins: regulators of transcription in eukaryotic organisms. Molecular and Cellular Biology. 20: 429-440.
Needleman and Wunsch, J. Mol. Biol. 48: 443-453, 1970 Sambrook et al (eds). Molecular Cloning. A Laboratory Manual. Cold Spring Harbor Laboratories, Cold Spring Harbor, N.Y., USA, 1989
Schafer, D. F. and Sorrell, M. F., Lancet 363:1253-1257, 1999
Willis A E. 1999. Translational control of growth factors and proto-oncogene expression. The International Journal of Biochemistry and Cell Biology. 31: 73-86.

What is claimed is:

1. A method for diagnosing human hepatocellular carcinoma or a related condition in a subject or a propensity for said subject to develop human hepatocellular carcinoma or a related condition, said method comprising the step of
identifying expression of a gene which is differentially or preferentially expressed in tissue from subjects with hepatocellular carcinoma or a related condition relative to other tissue in said subject and/or subjects not diagnosed with this condition.

2. The method of claim 1, wherein the gene comprises a nucleotide sequence as set forth in SEQ ID NO:1 or SEQ ID NO:3 or a nucleotide sequence having at least about 60% similarity to SEQ ID NO:1 or SEQ ID NO:3 after optimal alignment or a nucleotide sequence capable of hybridizing to SEQ ID NO:1 or SEQ ID NO:3 under low stringency conditions.

3. The method of claim 2, wherein the gene comprises a nucleotide sequence as set forth in SEQ ID NO:1 or SEQ ID NO:3 or a nucleotide sequence that hybridizes to SEQ ID NO:1 or SEQ ID NO:3 under high stringency conditions which include from 31% v/v to 50% v/v formamide and from 0.01 M to 0.15 M salt for hybridization and from 0.01 M to 0.15 M salt for washing.

4. A modulator of expression of a nucleic acid molecule, which nucleic acid molecule is differentially or preferentially expressed in human hepatocellular carcinoma tissue or tissue from a related cancer relative to other tissue in said subject and/or in subjects not diagnosed with this condition, that decreases the expression of said nucleic acid molecule.

5. The modulator of expression of claim 4, wherein the modulator is an antisense molecule.

6. The modulator of expression of claim 4, wherein the nucleic acid molecule comprises a nucleotide sequence as set forth in SEQ ID NO:1 or SEQ ID NO:3 or a nucleotide sequence having at least about 60% similarity to SEQ ID NO:1 or SEQ ID NO:3 after optimal alignment or a nucleotide sequence capable of hybridizing to SEQ ID NO:1 or SEQ ID NO:3 under low stringency conditions.

7. A method of modulating one or more activities within a cell, said method comprising the step of
modulating expression of hcc-1 gene expression or the activity of HCC-1 for a time and under conditions sufficient to modulate the cell activity.

8. A method of treating hepatocellular carcinoma or a related condition, said method comprising the step of administering to a subject in need of such treatment an antagonist of a gene or gene product which is differentially or preferentially expressed in tissue from subjects with hepatocellular carcinoma or a related condition relative to other tissue in said subject and/or subjects not diagnosed with this condition.

9. The method of claim 8, wherein an antagonist of a gene, that comprises a nucleotide sequence as set forth in SEQ ID NO:1 or SEQ ID NO:3 or a nucleotide sequence having at least about 60% similarity to SEQ ID NO:1 or SEQ ID NO:3 after optimal alignment or a nucleotide sequence capable of hybridizing to SEQ ID NO:1 or SEQ ID NO:3 under low stringency conditions, is administered to said subject.

10. The method of claim 8, wherein an antagonist of a gene product, that comprises an amino acid sequence as set forth in amino acid sequence SEQ ID NO:2 or an amino acid sequence having at least 60% similarity to SEQ ID NO:2 after optimal alignment or an amino acid sequence encoded by the nucleotide sequence set forth in SEQ ID NO:1 or SEQ ID NO:3 or a nucleotide sequence having at least about 60% similarity to SEQ ID NO:1 or SEQ ID NO:3 after optimal alignment or a nucleotide sequence capable of hybridizing to SEQ ID NO:1 or SEQ ID NO:3 under low stringency conditions, is administered to said subject.

11. An isolated peptide, polypeptide, or protein, or a derivative, homologue, or analogue thereof, which protein is differentially or preferentially produced in or by human hepatocellular carcinoma tissue or tissue from a related cancer relative to other tissue in said subject and/or in subjects not diagnosed with this condition.

12. The isolated peptide, polypeptide, or protein of claim 11, comprising
the amino acid sequence of SEQ ID NO:2 or
an amino acid sequence having at least 60% similarity to SEQ ID NO:2 after optimal alignment or
an amino acid sequence encoded by the nucleotide sequence of SEQ ID NO:1 or SEQ ID NO:3 or a nucleotide sequence having at least about 60% similarity to SEQ ID NO:1 or SEQ ID NO:3 after optimal alignment or a nucleotide sequence capable of hybridizing to SEQ ID NO:1 or SEQ ID NO:3 under low stringency conditions.
